(19) United States
(12) Patent Application Publication
(10) Pub. No.: US 2015/0154373 A1
(43) Pub. Date: Jun. 4, 2015

(54) DISEASE RISK DECISION SUPPORT PLATFORM

(71) Applicant: Charis Eng, Cleveland Heights, OH (US)

(72) Inventor: Charis Eng, Cleveland Heights, OH (US)

(73) Assignee: THE CLEVELAND CLINIC FOUNDATION, Cleveland, OH (US)

(21) Appl. No.: 14/611,792

(22) Filed: Feb. 2, 2015

Related U.S. Application Data
(63) Continuation of application No. 13/655,816, filed on Oct. 19, 2012.
(60) Provisional application No. 61/549,278, filed on Oct. 20, 2011.

(51) Int. Cl. G06F 19/00; A61B 5/00

(52) U.S. Cl. CPC G06F 19/3431; A61B 5/7275; A61B 5/486; A61B 5/7264; A61B 5/742; A61B 5/7475; G06F 19/363

(57) ABSTRACT

A system provides a disease specific risk reference and includes a plurality of executable item modules that each define a different elementary disease to family structure relationship for a specific disease represented as a logical Boolean operation, and an item scoring engine that ranks positively scored item modules based on a risk level associated with a corresponding elementary disease to family structure relationship, wherein the positive scores identify an existence of a given disease to family structure relationship. The system further includes a disease specific risk reference generator that extracts item content associated with a subset of the highest ranked positively scored item modules from memory and provides the extracted item content in a disease specific risk reference for review by a clinician.

### CROSS-REFERENCE TO RELATED APPLICATION

This application claims the benefit of U.S. Provisional Patent Application No. 61/549,278, filed Oct. 20, 2011, and entitled CLINICAL DECISION SUPPORT PLATFORM, the entire contents of which is incorporated herein by reference.

### TECHNICAL FIELD

This disclosure relates to health care and more particularly to a disease risk decision support platform.

### BACKGROUND

Family members share genes, behaviors, lifestyles, and environments that together may influence their health and their risk of chronic disease. Most people have a family health history of some chronic diseases (e.g., cancer, coronary heart disease, and diabetes) and health conditions (e.g., high blood pressure and hypercholesterolemia). People who have a close family member with a chronic disease may have a higher risk of developing that disease than those without such a family member. Family health history is a written or graphic record of the diseases and health conditions present in an individual's family. For example, a useful family health history shows three generations of biological relatives, the age at diagnosis, and the age and cause of death of diseased family members. Family health history is a useful tool for understanding health risks and preventing disease in individuals and their close relatives.

Many genetic disease risk assessment tools for clinicians are based on a single or a few complex clinical guidelines and/or published risk assessments derived from published literature and studies. From these guidelines, complex verbatim statements are used, for example, in a patient's chart to provide a clinician with guidance on whether a patient is at risk for a given specific disease and needs further treatment and/or evaluation. The verbatim statements are typically difficult to understand, can be counter intuitive, lack consistent logical structure or syntax, and in some cases can contradict one another. Therefore, the verbatim statements can be difficult for even the most seasoned clinician to consistently understand and apply.

### SUMMARY

This disclosure relates to health care and more particularly disease risk decision support platform systems and methods.

As one example, a system for providing a disease specific risk reference is disclosed. The system comprises a plurality of executable item modules that each define a different elementary disease to family structure relationship for a specific disease represented as a logical Boolean operation, and an item scoring engine that ranks positively scored item modules based on a risk level associated with a corresponding elementary disease to family structure relationship, wherein the positive scores identify an existence of a given disease to family structure relationship. The system further comprises a disease specific risk reference generator that extracts item content associated with a subset of the highest ranked positively scored item modules from memory and provides the extracted item content in a disease specific risk reference for review by a clinician.

In another example, a non transitory computer readable medium is provided that stores instructions for performing a method. The method comprises receiving family structure information and family disease history responses to family disease history questions for a specific disease, executing a plurality of item modules that each define a different elementary disease to family structure relationship represented as a logical Boolean operation for a specific disease based on the family structure information and the family disease history responses, and ranking positively scored item modules based on a risk level associated with a corresponding elementary disease to family structure relationship, wherein the positive scores identify an existence of a given disease to family structure relationship. The method further comprises selecting a disease risk category based on a positively scored item module ranked with the highest risk ranking, wherein the disease risk level category rates a patient's level of risk for the specific disease. The method further comprises extracting item content associated with a subset of the highest risk positively scored item modules, and providing the extracted item content and the selected disease risk category in a disease specific risk reference for review by a clinician.

In yet another example, a computer-implemented method is provided. The computer-implemented method comprises receiving an appointment specific information request that has an association with one or more specific diseases, providing patient general and personal health history questions, providing family structure questions, and providing family disease specific history questions. The method further comprises performing an intermediate scoring of one or more family disease history to family structure relationships based on answers to the family disease specific history questions, and providing family member disease specific history questions for each family member identified with a disease specific history that had an intermediate score that exceeded a predetermined threshold.

### BRIEF DESCRIPTION OF THE DRAWINGS

FIG. 1 depicts an example block diagram of a disease risk decision platform system that can be implemented according to an embodiment.

FIG. 2 depicts an example block diagram of a clinical development methodology that can be implemented to derive item modules and associated questions according to an embodiment.

FIG. 3 illustrates an example of questions derived from an interpreted statement.

FIG. 4 illustrates an example of a sample algorithm derived from the same interpreted statement and executed based on the responses from FIG. 3, and a table based on the information provided by the patient including the responses to the questions in FIG. 3.

FIG. 5 depicts an example of a disease risk decision reference system interaction diagram.

FIG. 6 illustrates a computer implemented method for generating a disease specific questionnaire in accordance with an embodiment.

FIGS. 7-14 illustrates various examples of a personal health questions, family structure entry and maintenance, and family health disease history questions associated with a given questionnaire displayed in a patient input/output (I/O) question and answer (Q&A) graphical user interface (GUI).

FIG. 15 illustrates a methodology 200 for generating a disease specific risk reference in accordance with an embodiment.

FIGS. 16-17 illustrate an example of a disease specific risk reference in accordance with an embodiment.

FIG. 18 illustrates a disease specific pedigree image that corresponds to the responses and family structure information provided in some of the answers to the example question of FIGS. 7-14.

FIG. 19 depicts an example of a computer architecture in which a disease risk reference generation system can be implemented according to an embodiment.

### DETAILED DESCRIPTION

This disclosure relates to health care and more particularly to a disease risk decision platform.

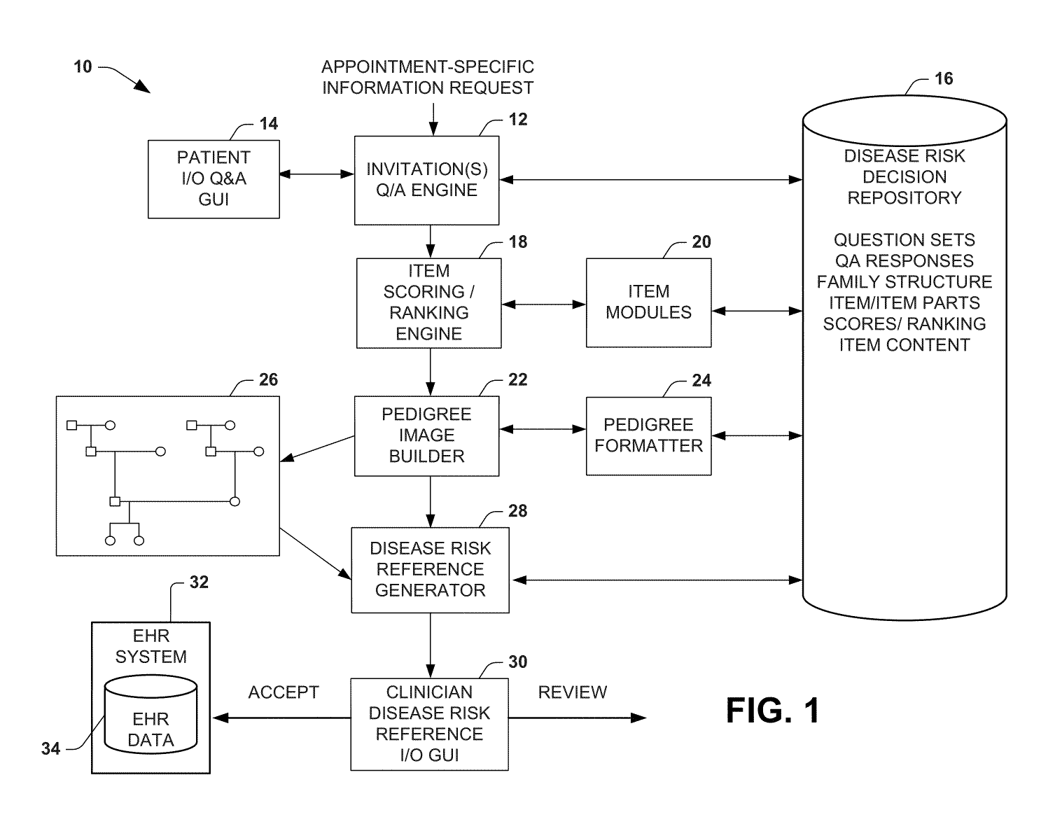

FIG. 1 depicts an example of a disease risk decision platform system 10 that can be implemented according to an embodiment. The system 10 is configured to generate disease specific risk reference information and guidelines to facilitate a clinician decision making process in diagnosing and providing recommendation to patients for further evaluation and/or specific treatments and/or life style behavior changes in accordance with disease risk. The system 10 employs patient family structure responses and patient genetic disease history responses to generate one or more disease specific pedigree data structures. The patient family structure and patient disease history responses are employed as input to a plurality of executable item modules 20 that each define a set of elementary disease to family structure relationships that can be represented as a logical Boolean operation. For example, for a given item module, an item module may be true if a relationship exists of a first degree relative having a disease X between an age range Y-Z, and a second first degree relative of the patient having disease X between the age range Y-Z. The given item module is then determined to be true and assigned a positive score and then ranked based on its preassigned disease risk category and/or risk level associated with the item module. An iterative process through all item modules is executed.

For example, this item module may be assigned to one of the following disease risk categories “Genetic Risk Present”, which is considered a very high risk, “Familial Risk”, which is considered a high risk, “Raised Risk”, which is considered a medium risk, and “Population Risk”, which is considered a low risk. Each item module is derived from a simple interpreted statement that is derived from a plurality of clinical guidelines and/or published risk assessments for a given disease, which will be explained further below. Each interpreted statement is also employed to derive an associated question or set of questions corresponding to a given item module. Each item module can also have an associated disease risk level within a disease risk category. For example, one item module may be ranked “Genetic Risk Present” based upon published evidence of the ways the patterns of disease manifest within the family. Each item module would have a relative rank given the pattern of disease being expressed in the interpreted statement and what evidence is published about the associated risk of such a pattern of disease within the family.

The disease risk decision platform system 10 includes an invitation question and answer (Q&A) engine 12 and a patient Input/Output (I/O) Q&A graphical user interface (GUI) 14. The invitation rules Q&A engine 12 receives an appointment-specific information request from a patient master scheduling center. The appointment-specific information request initiates one or more invitation requests based on the specific appointment made by the patient. Each invitation request is related to the patient's scheduled encounter and invokes a question set or disease-related questionnaires including, for example, patient general questions, patient personal health history questions, family structure questions, and family disease specific history questions. For example, an appointment for a specialist may initiate a single question set or questionnaire, while a well check can encompass multiple question sets.

The invitation Q&A engine 12 retrieves question sets associated with the one or more invitations from a disease risk decision repository 16 and delivers a questionnaire to the patient I/O Q&A GUI for receiving answers from the patient. The invitation Q&A engine 12 stores the patient's responses to the questions in the disease risk decision repository 16. The invitation Q&A engine 12 can be configured to suppress redundant questions when providing multiple invitation question sets, such that the patient does not need to answer the same question multiple times. Furthermore, the invitation Q&A engine 12 can be configured to employ branch logic and filtering, such that additional questions can be added to the question set based on answers received from the patient, or that specific questions can be provided (e.g., female directed questions) and specific question can be omitted (e.g., male directed questions) based on answers received by the patient.

Additionally, the disease-specific questions can be intermediately scored to determine if they exceed a certain risk threshold, which could cause the invocation of additional questions to be provided to the patient. Once the questionnaire is completed and submitted by the patient, the set of answers are provided as disease related responses and family structure data or information to an item scoring/ranking engine 18. The term answers will be referred to as encompassing all answers to questions in a questionnaire, while the term responses will be referred to as answers to disease specific questions and family structure questions employed by the executable item modules 20.

The item scoring/ranking engine 18 executes a set of item modules of the plurality of item modules 20 associated with the specific disease employing the patient's responses and family structure information associated with the specific disease being evaluated. Each item module that provides an indication of a presence of a disease to family structure relationship can be scored as a positive scored item, while each item module that provides an indication of the absence of a given disease to family structure relationship can be scored as a false or negative scored item. Each positive scored item module is ranked based on the highest disease risk levels associated with the positive scored items. Based on the highest ranked disease risk positively scored item or items, item content (e.g., disease risk level category, recommendations, assessments, diagnosis codes, education links, disease overview, etc.) that resides in the disease risk decision repository 16 is identified for providing to a disease specific risk reference. Each item module may have multiple parts (e.g., up to 3 item parts). In the present example, an item module can be considered positive or true if each item module is positive or true. However, it is to be appreciated that other scoring methodologies can be employed.

The disease specific responses and family structure information can be formatted by a pedigree formatter 24. The formatter 24 formats the disease related responses and the family structure information in a format (e.g., XML) to be read by a pedigree image builder 22 that can generate a disease specific pedigree image 26 related to the specific disease being evaluated.

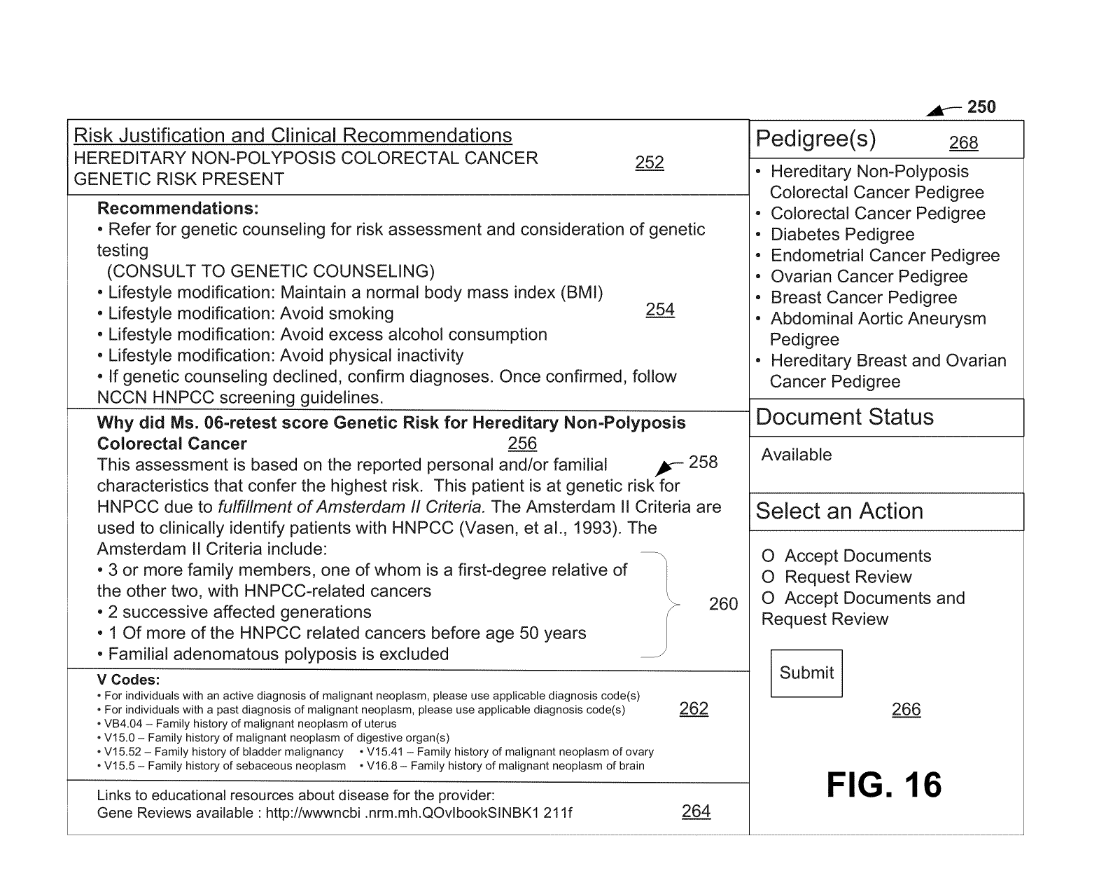

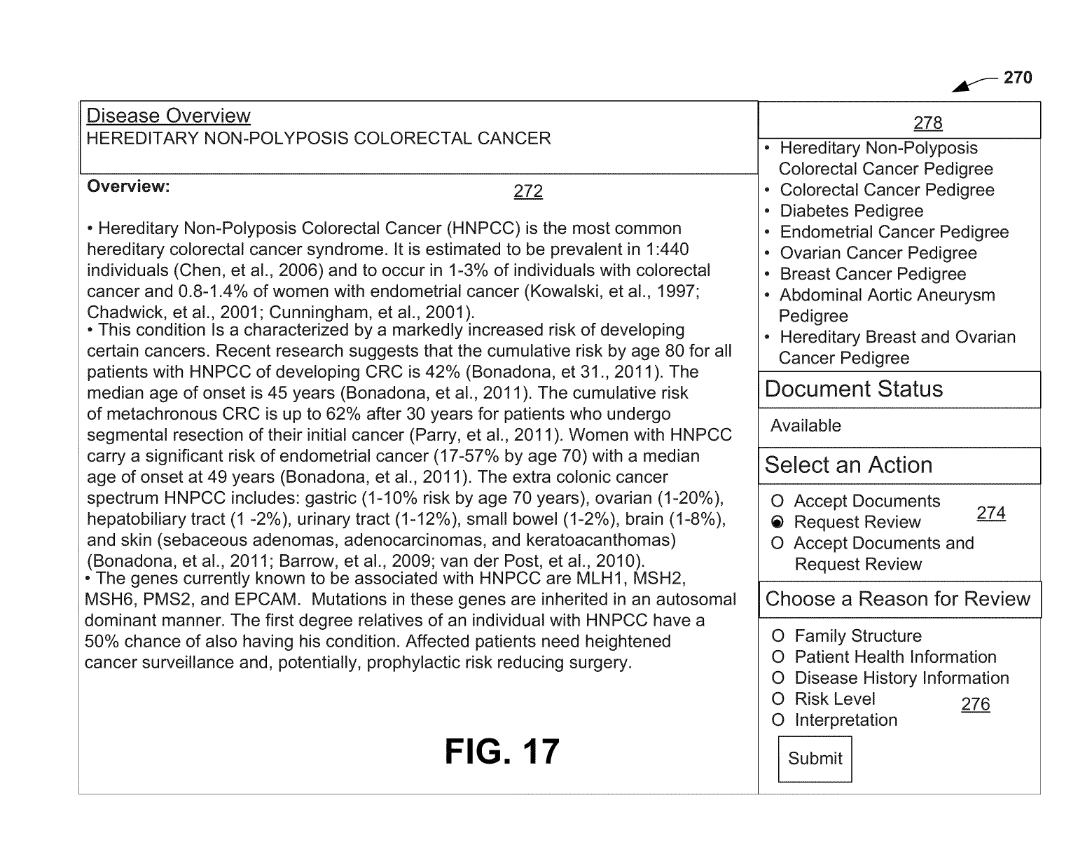

A disease specific risk reference generator 28 is configured to access the disease specific pedigree image to be provided to a clinician disease specific risk reference I/O GUI 30. Additionally, the disease specific risk reference generator 28 accesses formats and displays the associated item content in the clinician disease specific risk reference I/O GUI 30 as a disease specific risk reference (see FIGS. 16-17) having one or more pages, for example, in PDF or HTML formatted page(s), for clinician review. Links may be provided for the clinician to view different disease specific risk reference pages and associated disease specific pedigree images. The disease specific risk reference may include various section of text associated with the item content of the identified positive scored and higher risk ranked item modules, such as text of the specific disease being evaluated, a disease risk level category of the patient for the specific disease, a recommendation section for providing recommendations for the patient such as lifestyle and/or medication changes, an assessment section that displays the reasons for the displayed disease risk level category, diagnosis codes for billing purposes and/or diagnosis, education links on the specific disease, disease overview, etc.

The assessment section can display textual versions of the elementary interpreted statements associated with the higher ranked positively scored item modules. After review of the disease specific risk reference page or pages by the clinician, the clinician can choose to accept the disease specific risk reference, which results in the storing of the disease specific risk reference evaluation in an electronic health record database 34 in an electronic health record system 32. Alternatively or additionally, the can choose to have the disease specific risk reference reviewed by a genetic expert or counselor. The alternative paths could repeat until a terminating action of acceptance by the clinician.

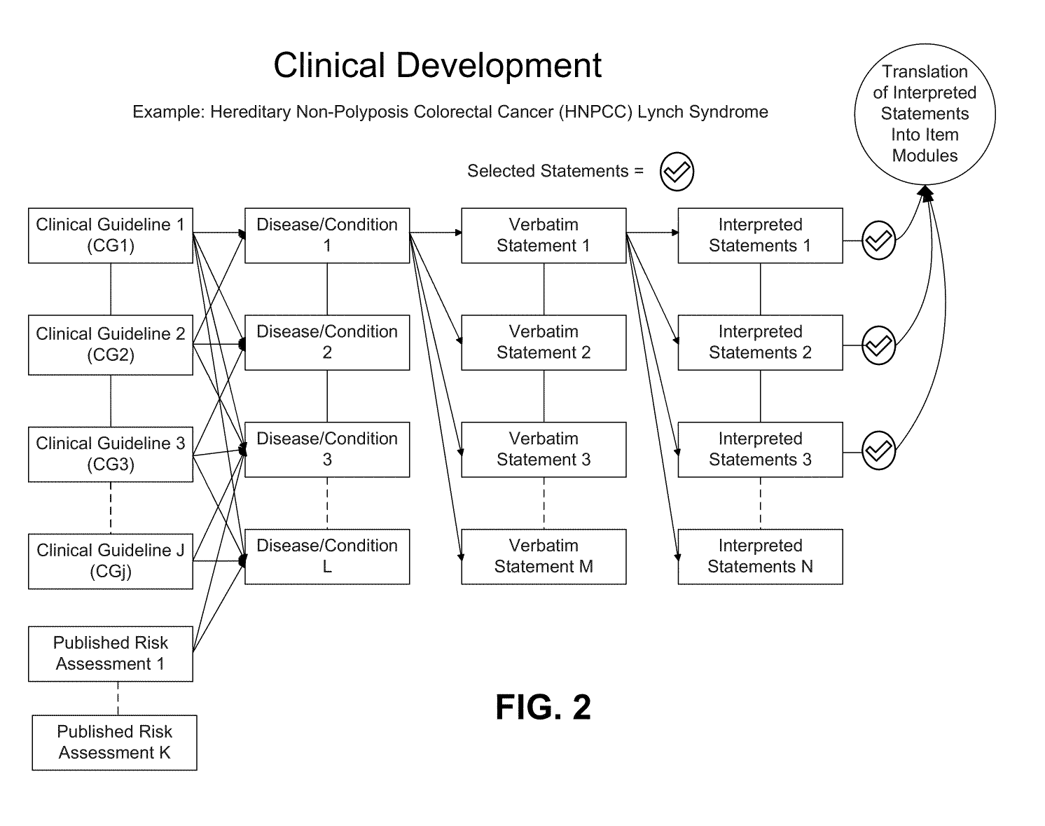

As stated above, each item module is derived from a simple interpreted statement that is derived from a plurality of clinical guidelines and/or published risk assessments for a given disease. FIG. 2 illustrates a clinical development process employed to derive interpreted statements and item modules in accordance with a specific disease example. A plurality of published clinical guidelines 1 through J and/or a plurality of published risk assessments 1 through K are reviewed to determine a plurality of disease/conditions relationships 1 through L, where J, K and L are integers greater than one. Each disease/condition can be determined from a number of published clinical guidelines and risk assessments. Some examples of a disease/condition can be colorectal cancer, endometrial cancer, synchronous or metachronous colorectal cancer, gastric cancer, personal and family history of HNPPC-related cancer Inflammatory bowel disease, etc.

The present example is for Hereditary Non-Polyposis Colorectal Cancer (HPNCCC) Lynch Syndrome. In such an example, 20 disease/conditions can be determined from seven clinical guidelines and four risk assessments. A plurality of complex verbatim statements labeled 1 through M are determined for each of the plurality of disease/conditions. A plurality of interpreted statements labeled 1 through N can then be derived from the plurality of complex verbatim statements, where M and N are integers greater than one. For example, 200 or more verbatim statements can be derived from the 20 disease/conditions, and 2000 or more interpreted statements can be derived from the 200 or more verbatim statements. A subset of the interpreted statements can then be selected to be employed to derive a plurality of item modules and associated disease to family structure related questions.

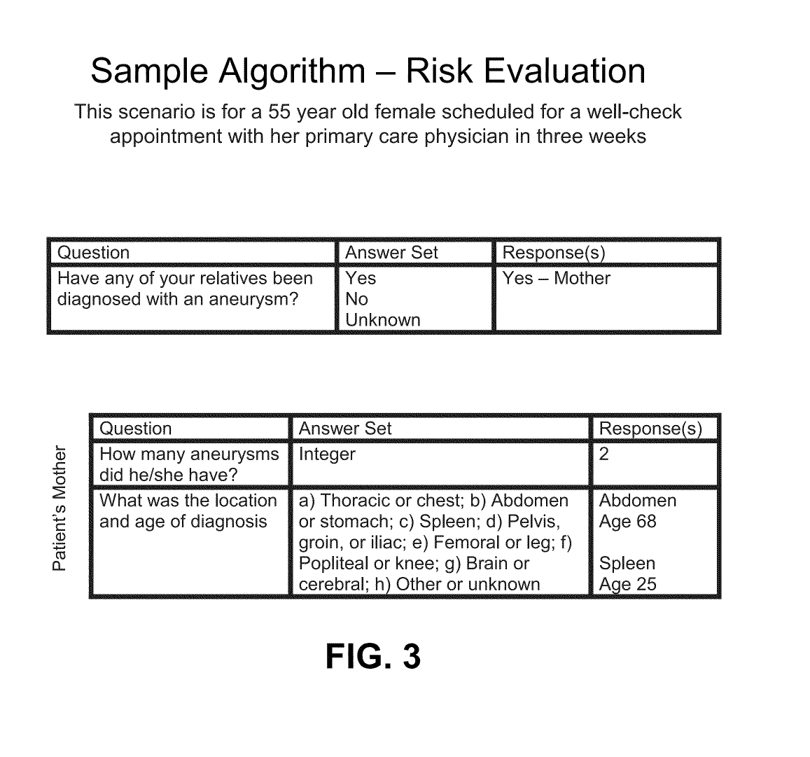

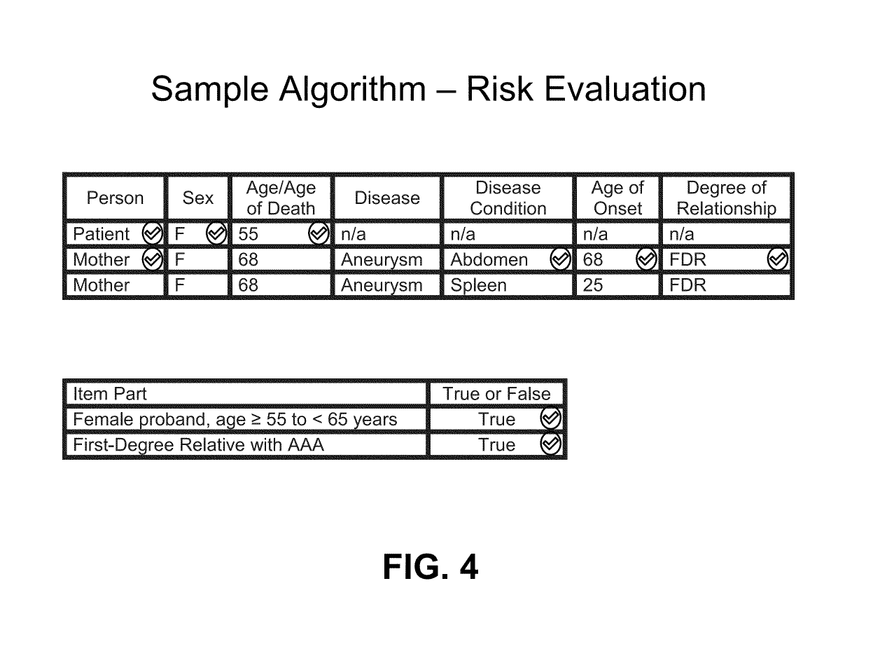

FIG. 3 illustrates an example of questions derived from an interpreted statement. The interpreted statement may be, for example, a female patient whose age is greater than or equal to 55 but less than 65 with a first degree relative who was diagnosed with an abdominal aortic aneurysm (AAA) is herself at a high or “Familial Risk” risk of AAA. A first question set is provided to the patient asking whether any relatives have been diagnosed with an aneurysm. The patient has provided an affirmative answer indicating that the mother has had at least one aneurysm. In response to the first question set, a second question set is provided that ask further questions about the mother's aneurysm. For example, a first question of the second question set is provided that asks the patient how many aneurysm did the mother have followed by second question of the question set that asks what was the location and age of the diagnosis. FIG. 4 illustrates an example of a sample algorithm derived from the same interpreted statement and executed based on the responses from FIG. 3. FIG. 4 illustrates a table based on the information provided by the patient including the responses to the questions in FIG. 3. FIG. 4 also illustrates the logical Boolean item part operations associated with the item module corresponding to the interpreted statement. As is shown in FIG. 4, both item parts are true, therefore, the item module is true.

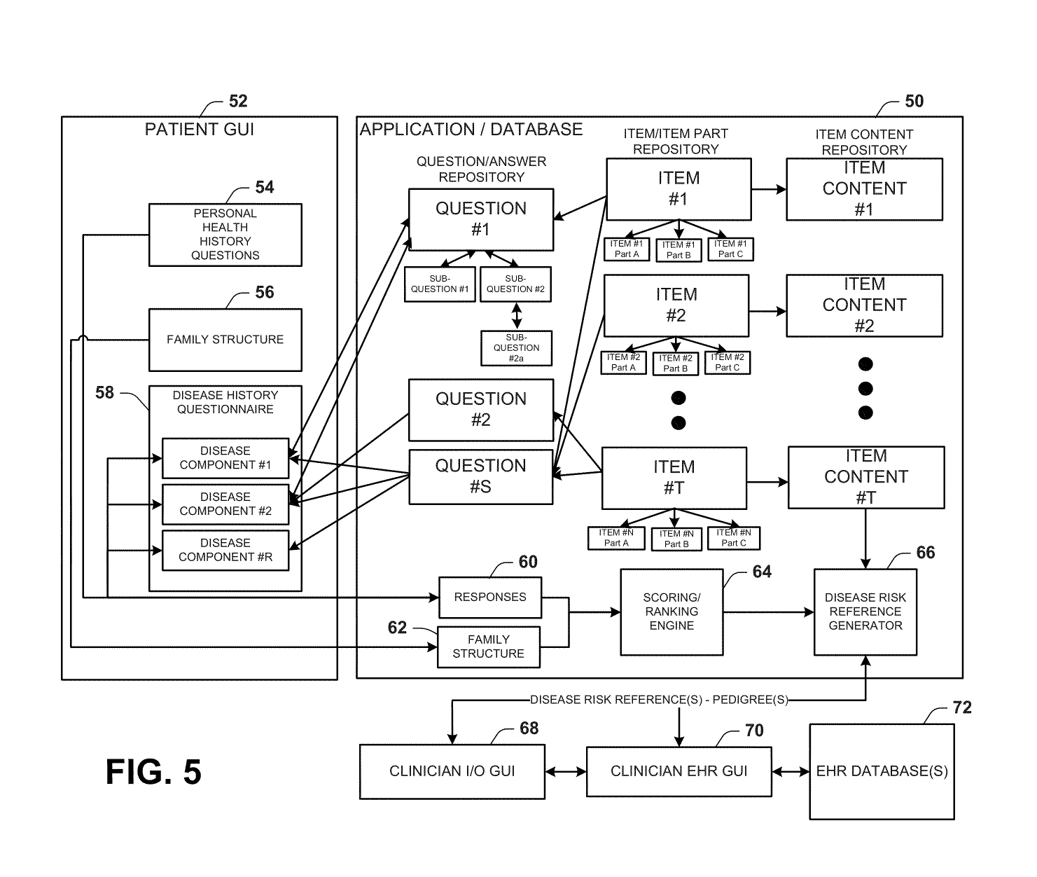

FIG. 5 depicts an example of a disease risk reference/decision support system interaction diagram. The interaction diagram illustrates the interaction and association between components associated with the generation of a disease risk reference and the patient and clinician I/Os. As illustrated in FIG. 5, the disease risk reference components 50 includes both application executable code and data stored in a database. Personal health history 54, family structure 56 and disease history questions 58 are provided to a patient I/O Q&A GUI 52 in response to a patient appointment specific information request. The disease history questions 58 include disease specific components labeled #1-#R that include one or more specific diseases questions or question sets labeled #1 through #S, where R and S are integers greater than or equal to one. It is to be appreciated that different disease components can include some of the same questions or question sets as other disease components.

Each disease specific question corresponds to at least one item module, labeled #1-#T, and each item module corresponds to item content also labeled #1-#T, where T is an integer greater than one. The disease specific question can include multiple sub-question parts that solicit responses that correspond to multiple item parts found in the related item module. In the present example, question #1 indicates a sub-question #1 and sub-question #2 and a sub-question #2a of sub-question #2, while the corresponding item module includes 3 item parts shown as Item Part #1 A, Item Part #1 B, and Item Part #1 C. Disease history responses 60 and family structure information 62 are provided from the patient I/O Q&A GUI 52 to a scoring/ranking engine 64.

The scoring/ranking engine 64 can invoke the execution of the plurality of item modules associated with the specific diseases being evaluated and employing the patient's responses and family structure information. Each positive scored item module is ranked based on the highest disease risk levels associated with the positive scored items. Based on the highest ranked disease risk positively scored item or items, item content associated with those item modules is provided to a disease risk reference generator 66 for generation of a disease risk reference to be provided in one or both of a clinician I/O GUI 68 and a clinician EHR GUI 70. The clinician can accept the disease risk reference to be stored along with other patient information in an EHR database 72.

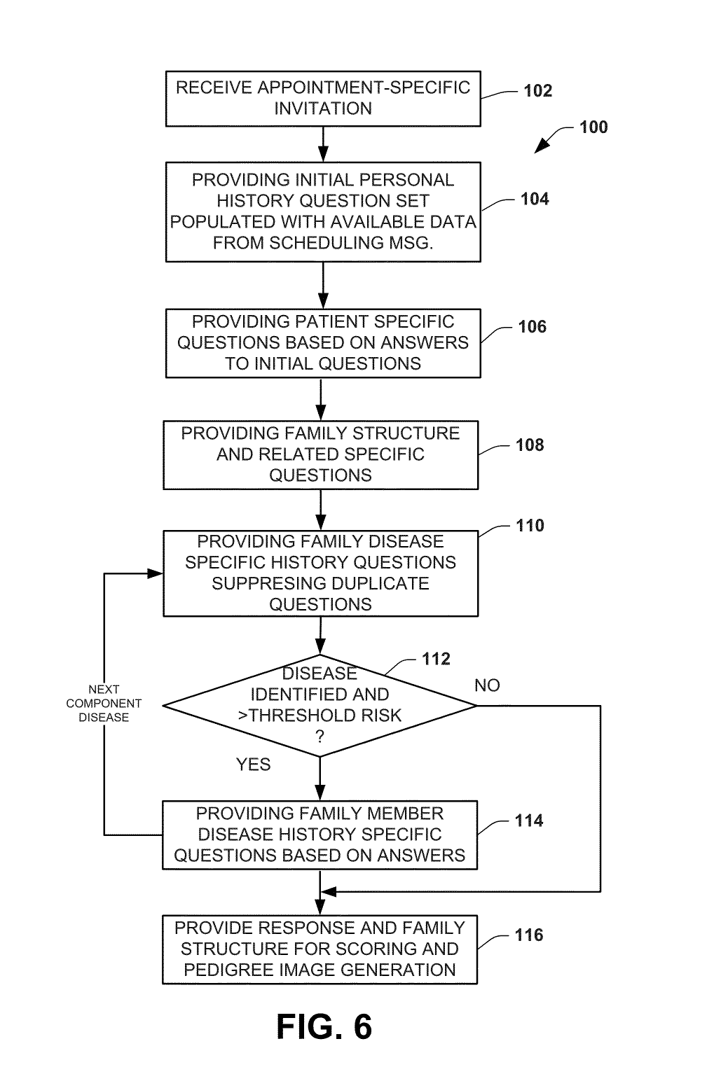

FIG. 6 illustrates a computer implemented method 100 for generating a disease specific questionnaire in accordance with an embodiment of the invention. The method begins at 102 where an appointment-specific information request is received, for example, from a patient master scheduling center. Each appointment-specific request can invoke one invitation associated with one or more specific diseases. The methodology will proceed to 104, where an initial personal health history question set is provided to a patient via a patient I/O Q&A GUI that can include questions populated with answers populated with available data from the scheduling message. Additionally, the questions can be populated with answers from previously answered invitations. For example, the patient's name, sex, ethnicity, race, weight, life style habits and medication information can be pre-populated.

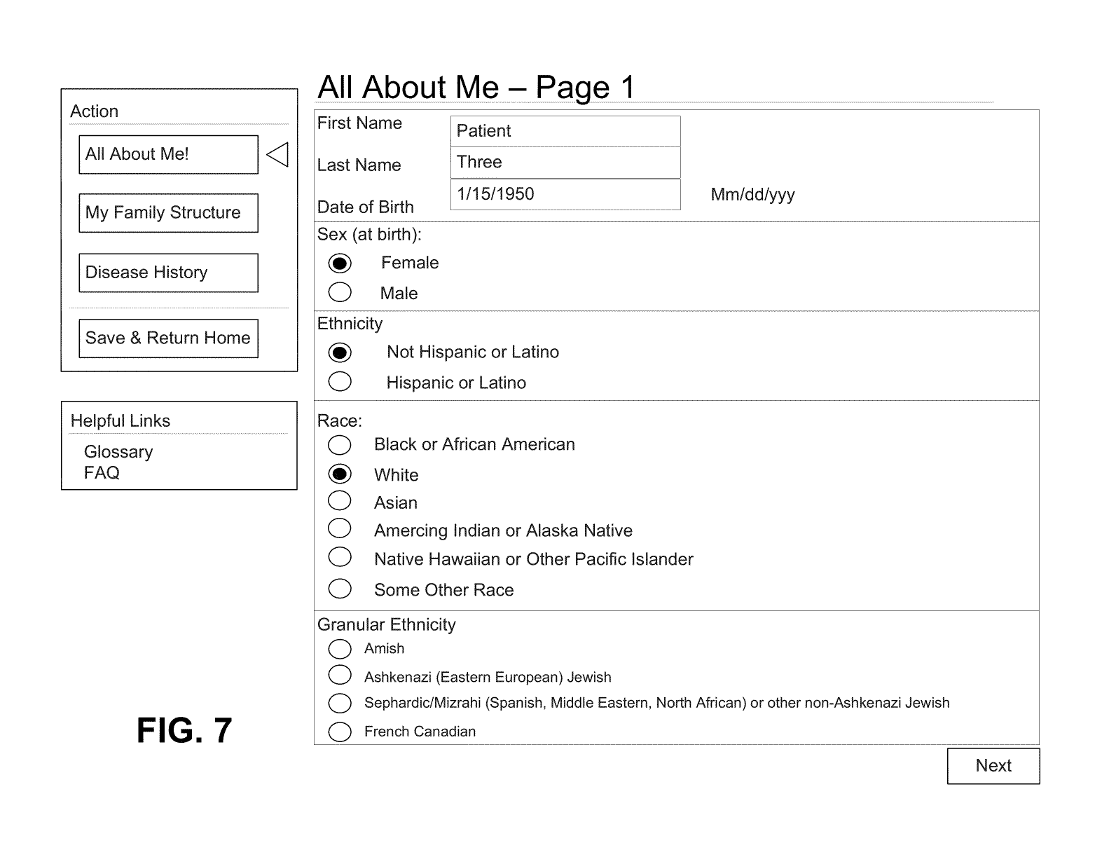

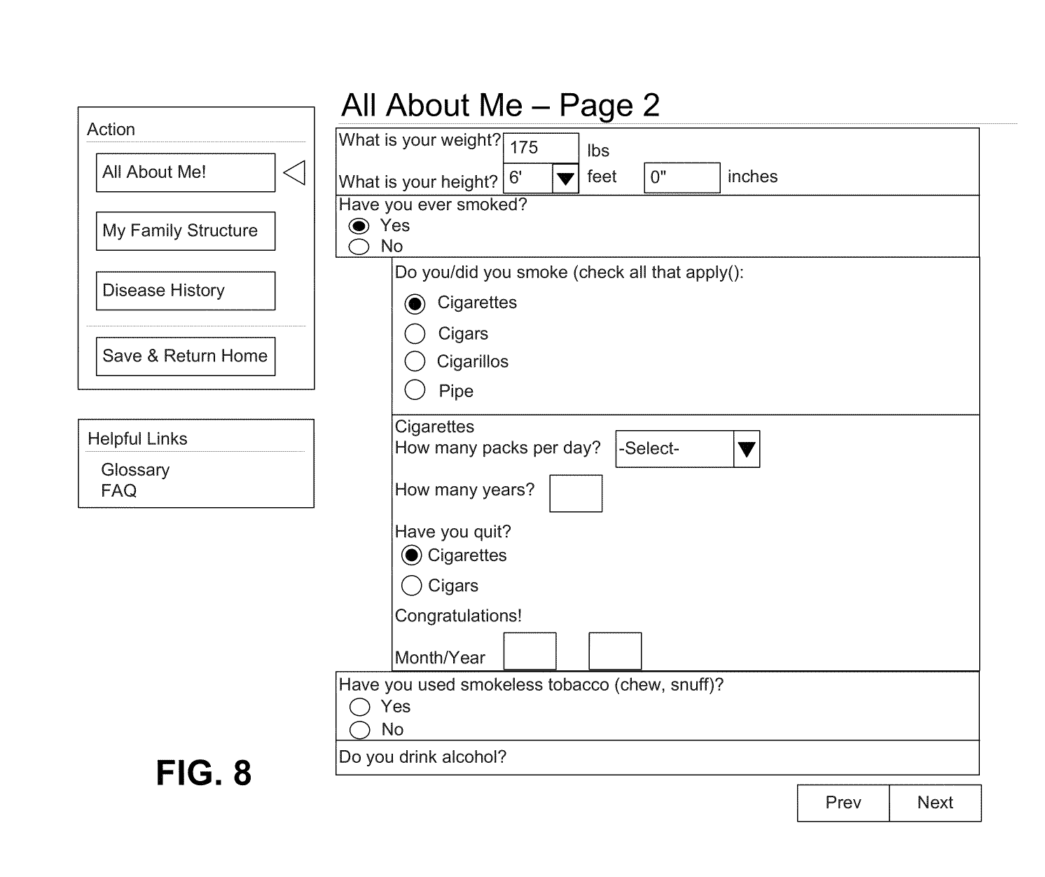

FIGS. 7-8 illustrate examples of questions from the personal health history question set provided in a patient I/O Q&A GUI. As illustrated in FIG. 7, basic patient information questions are presented such as name, sex, ethnicity, race and granular ethnicity. FIG. 8 illustrates the providing of basic health and health behavior information questions, such as height and weight of the patient, tobacco and alcohol consumption, and other item module relevant personal health questions. The patient can accept or modify the personal health information which is pre-populated if available in the scheduling message. The patient can complete the initial set of health behavior questions that were not pre-populated and click a “Next” button in the GUI.

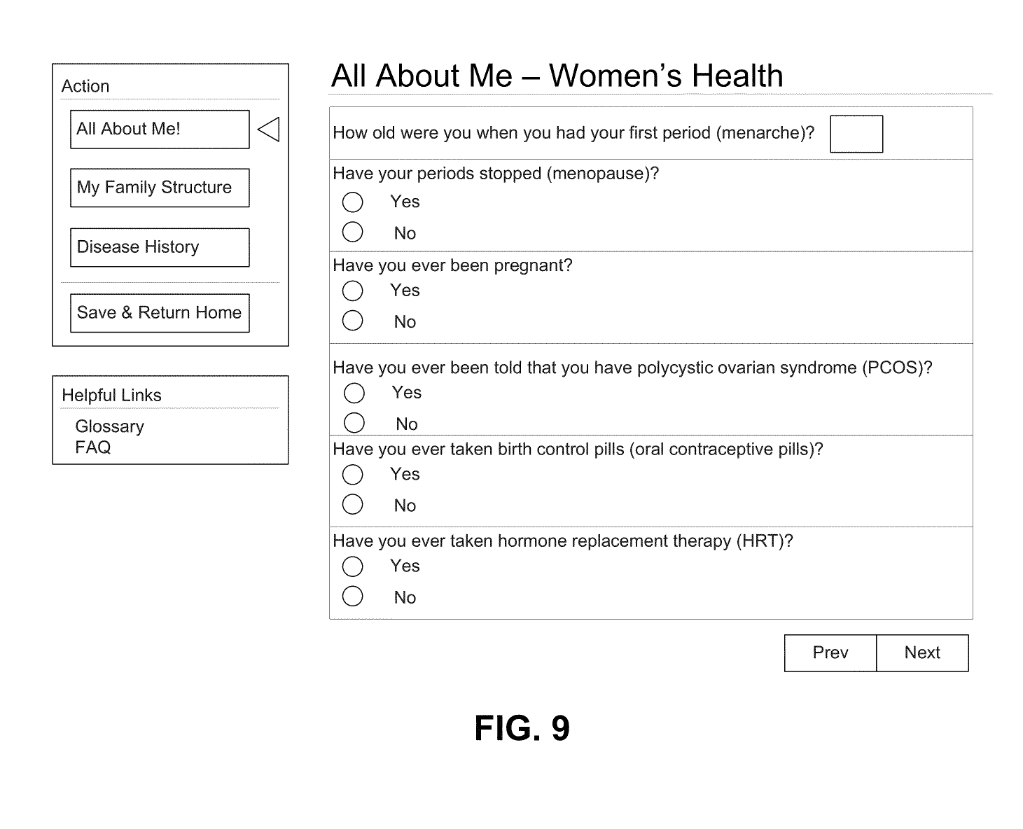

Referring again to FIG. 6, the methodology 100 then proceeds to 106 in response to receipt of a “NEXT” action response by the patient. At 106, the methodology provides patient-specific questions based on answers to initial questions. For example, the patient had identified themselves as a female patient in response to the sex question in FIG. 7. The methodology 100 then can provide female related questions and filter or suppress any male questions associated with the questionnaire. For example, FIG. 9 illustrates a patient I/O Q&A GUI with questions related to a women's period, whether they have ever been pregnant or not, whether they have been told they have polycystic ovarian syndrome (PCOS), whether have ever take birth controls or hormone replacement therapy (HRT). Upon completion of the patient specific questions, the patient can click a “FINISH” button to provide an indication that the patient specific general information has been completed.

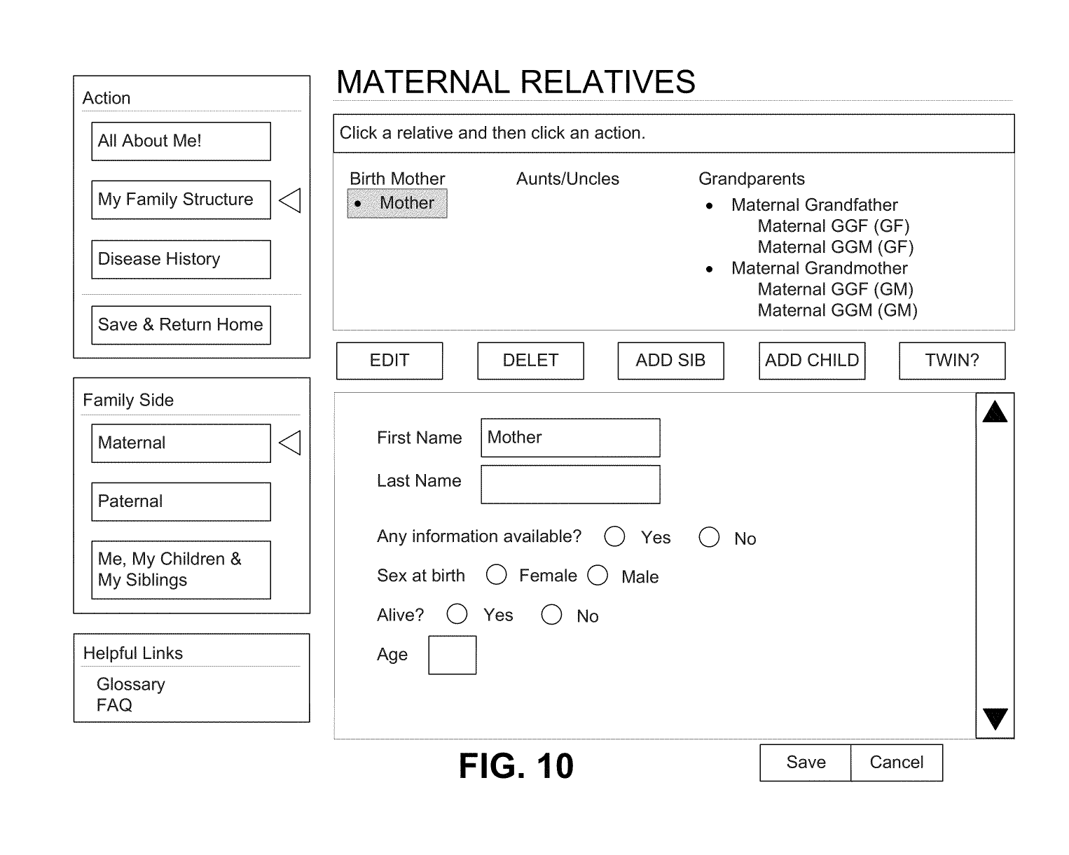

Referring again to FIG. 6, the methodology 100 then proceeds to 108 in response to receipt of a “FINISH” action response by the patient. At 108, the methodology provides family structure entry/maintenance GUI and related family structure questions to the patient. FIG. 10 illustrates a patient I/O Q&A GUI where a patient can provide information about family members (i.e., blood relatives). The patient can select a family side from the three side used to organize information including maternal side, paternal side, and the patient, patient's children and sibling side from a family side navigation box. A given family member can be selected in a selection box and information can be added for that family member, such as sex, whether or not the family member is alive and a family member's age. Once this is completed for each family member, the patient can select the “Next” button in the GUI.

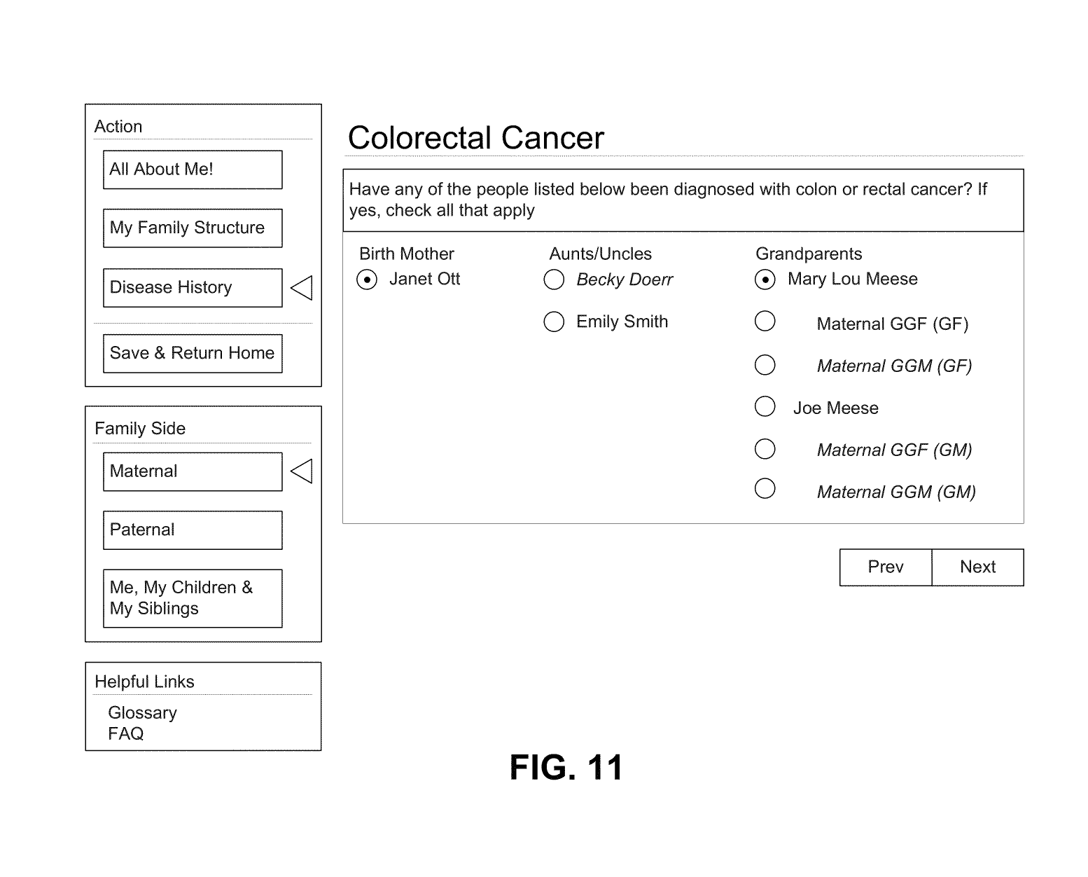

The methodology 100 of FIG. 6 then proceeds to 110 in response to the entry of family structure and receipt of family structure questions. At 110, the methodology 100 provides family structure disease specific questions to the patient. The disease specific questions can be based on the invitation requests associated with the appointment specific information request. FIG. 11 illustrates a patient I/O Q&A GUI where a patient can provide information about family disease history including information, which may also include history about the patient themselves. In the example, the patient is provided a questionnaire on whether there is a family history of any diagnosis of colorectal cancer. The patient has entered a diagnosis of colorectal cancer on the birth mother and grandmother on the maternal side. The patient can also complete the family history for the paternal side and patient/children/sibling side. Once this is completed for each, the patient can select the “Next” button in the GUI.

The methodology 100 of FIG. 6 then proceeds to 112 in response to receipt of patient-entered disease history responses. At 112, the methodology 100 evaluates the patient's risk for the diseases included in the invitation and determines if more disease history information should be collected. If a disease is identified that presents a potential risk that risk is greater than an established threshold risk (YES), the methodology proceeds to 114. If a disease is not identified that presents a potential risk, or an identified risk is determined that is not greater than a threshold risk (NO), the methodology proceeds to 116. The threshold risk can be determined by pre-scoring the family structure and patient responses to the disease specific history questions previously provided. Alternatively, the threshold risk determination can be eliminated, and any disease specific relationship identification may result in proceeding to 114.

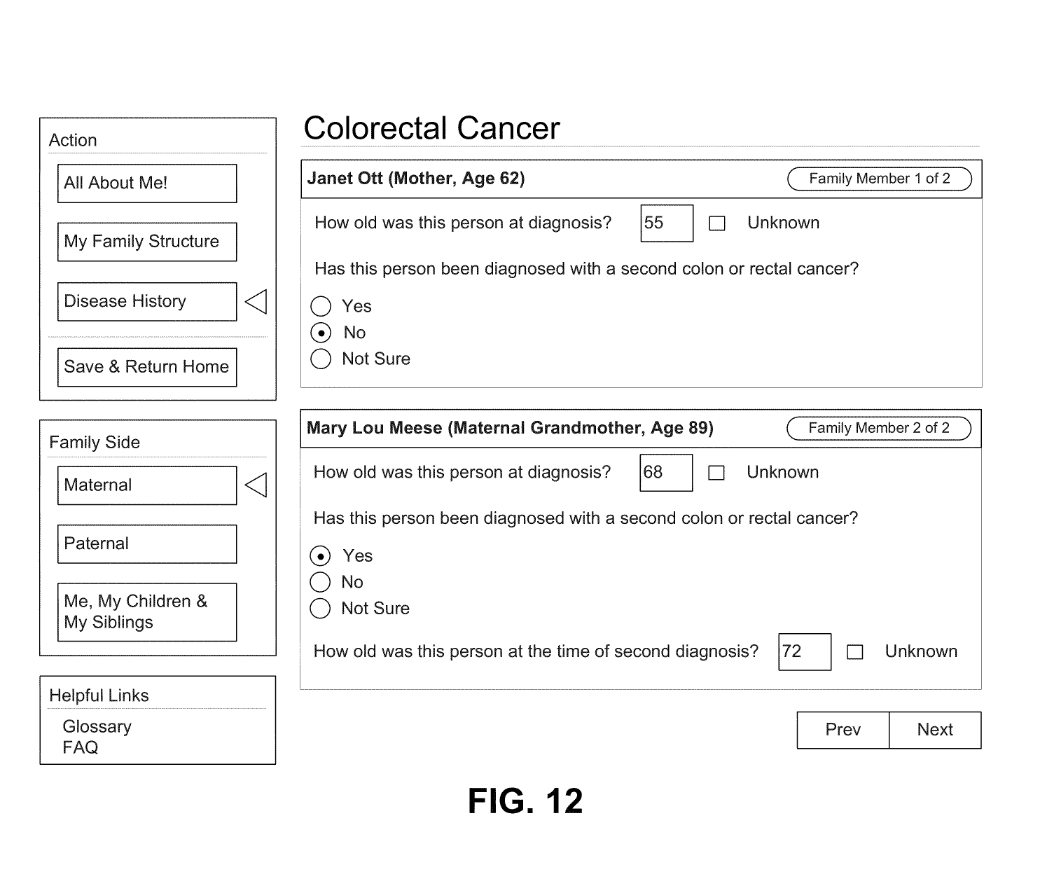

At 114, the methodology 100 provides family member disease specific history questions to the patient for each family member determined in 112 to be of risk interest. FIG. 12 illustrates a patient I/O Q&A GUI where a patient can provide answers to specific questions about each family member's disease history associated with the specific disease being evaluated. In the example of FIG. 12, the patient is provided a questionnaire asking how old the ages of the mother and grandmother when they were diagnosed with colorectal cancer, and whether either of them was diagnosed for colorectal cancer a second time. If either person did have a second diagnosis, the age of that diagnosis would be asked. Once this is completed for each, the patient can select the “Next” button in the GUI. FIG. 12 can be viewed as sub-questions #1, #2, and #2a of question #1 in FIG. 11, and discussed in FIG. 5.

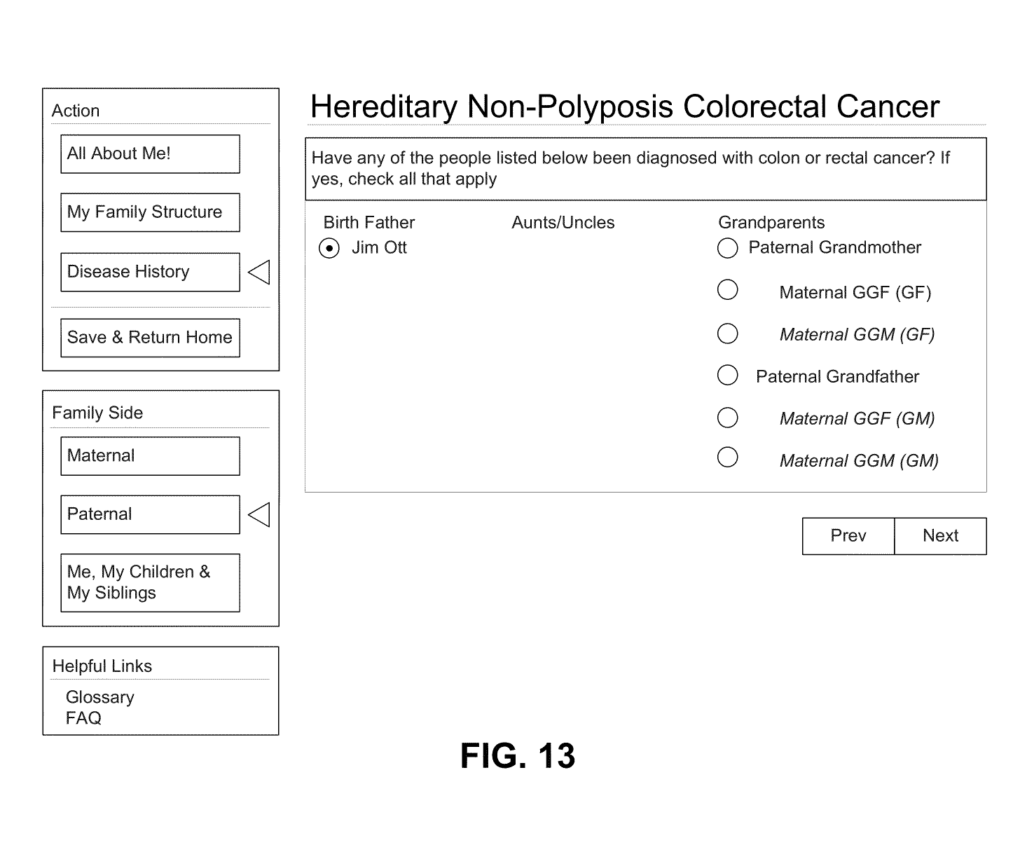

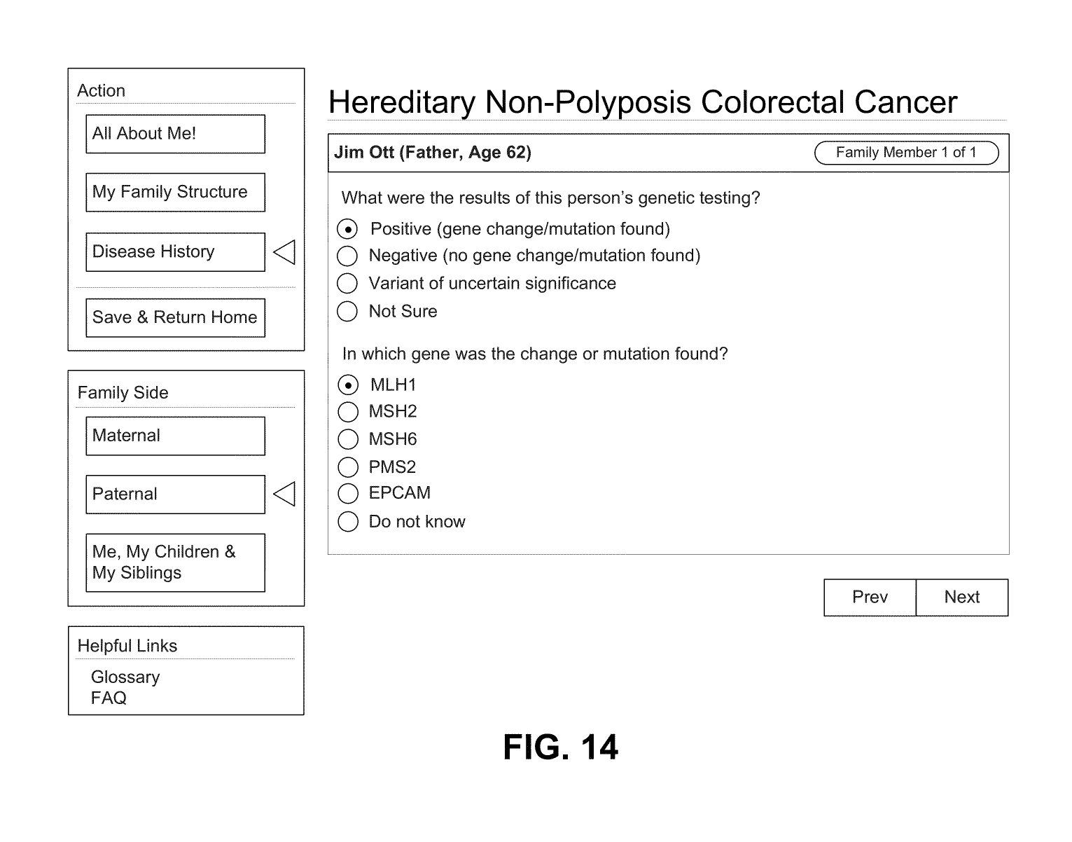

It is to be appreciated that different types of genetic disease histories can contribute to a given disease specific risk for a patient, especially for various types of cancer. Therefore, 110-114 may be repeated for additional disease components as illustrated by the dash line indicating a possible entry of a next component disease. For example, FIG. 13 illustrates a patient I/O Q&A GUI where a patient is now being asked whether there is any hereditary non-polyposis colorectal cancer in the family structure. The patient has entered that their birth father had been diagnosed with hereditary non-polyposis colorectal cancer. Upon selecting the “Next” button, FIG. 14 is displayed in a patient I/O Q&A GUI where the patient can provide answers to specific questions about the father's diagnosis of hereditary polyposis colorectal cancer. In the example of FIG. 14, the patient is provided a questionnaire on the results of the specific genetic testing and changed or mutated gene. Once this is completed for each, the patient can select the “Next” button in the GUI for the next disease component. Once all of the questions have been completed, the patient responses and family structure are provided for scoring and generation of a disease specific pedigree image at 116.

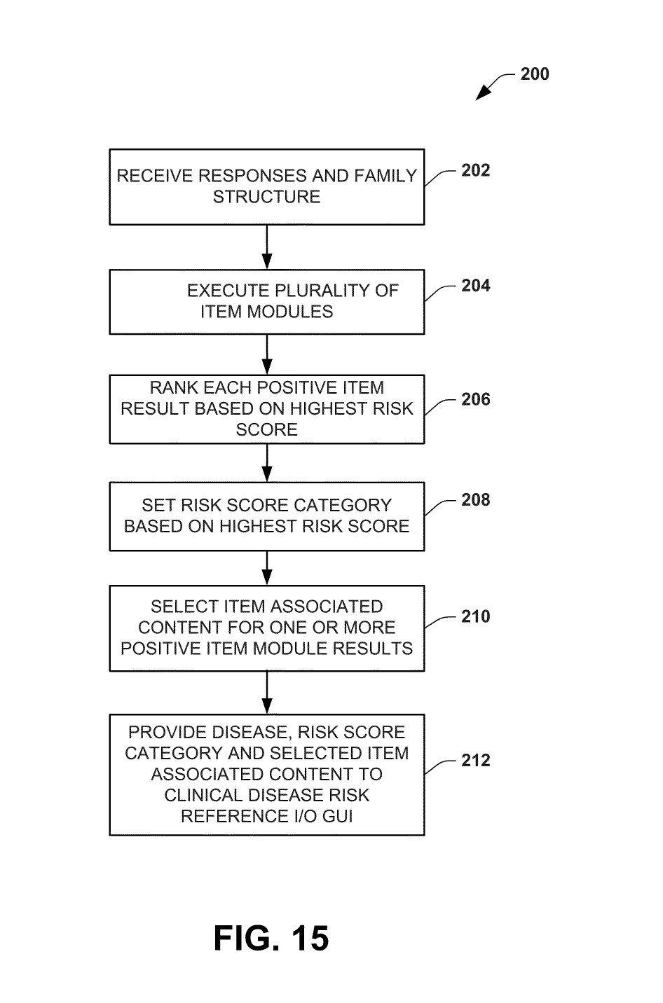

In this regard and in view of the foregoing structural and functional features described above, an example method will be better appreciated with reference to FIG. 15. While, for purposes of simplicity of explanation, the example method of FIG. 15 is shown and described as executing serially, the present examples are not limited by the illustrated order, as some actions could in other examples occur in different orders and/or concurrently from that shown and described herein. Moreover, it is not necessary that all described actions be performed to implement a method and other actions can be combined with those shown as disclosed herein. The example method of FIG. 15 can be implemented as computer-readable instructions that can be stored in a non-transitory computer readable medium such as can be computer program product. The computer readable instructions corresponding to the methods of FIG. 15 can also be executed by a processor.

FIG. 15 illustrates a methodology 200 for generating a disease specific risk reference in accordance with an embodiment. The methodology begins at 202 where response data and family structure data corresponding to a specific disease being evaluated is received. At 204, a plurality of item modules corresponding to the disease being evaluated are executed employing the response data and family structure data. At 206, each positive item module result is ranked based on its associated risk category and/or associated risk level. At 208, a subset (one or more) of the highest risk positive item modules are selected. At 210, item content associated with the subset of the highest risk item modules is extracted for the selected subset of the highest risk item modules. At 212, text associated with the disease being evaluated, the disease risk level category, and the selected item content is provided to a disease risk reference I/O GUI in one or more pages of a disease specific risk reference.

FIGS. 16-17 illustrate one example of pages from a disease specific risk reference in accordance with an aspect of the present invention. The disease specific risk reference evaluation includes a first page 250 with a header 252 that identifies the specific disease being evaluated (e.g., hereditary non-polyposis colorectal cancer) and the associated disease risk level category (e.g., genetic risk present) based on the highest risk positively scored item module. The disease specific risk reference includes a recommendation section 254 for providing a clinician with recommendations for the patient such as lifestyle and/or medication changes. The disease specific risk reference also includes an assessment section 256 that displays the reasons for the displayed disease risk level category. The reasons for the displayed disease risk level category can include text 258 describing the basis of the assessment and one or more text versions 260 of the interpreted statements associated with the one or more highly ranked positively scored item modules employed to determine the disease risk level category. The disease specific risk reference also includes diagnosis codes 262 (e.g., V Codes) for diagnostics and billing purposes, and education links 264 on the specific disease if the clinician desires to review literature about the specific disease.

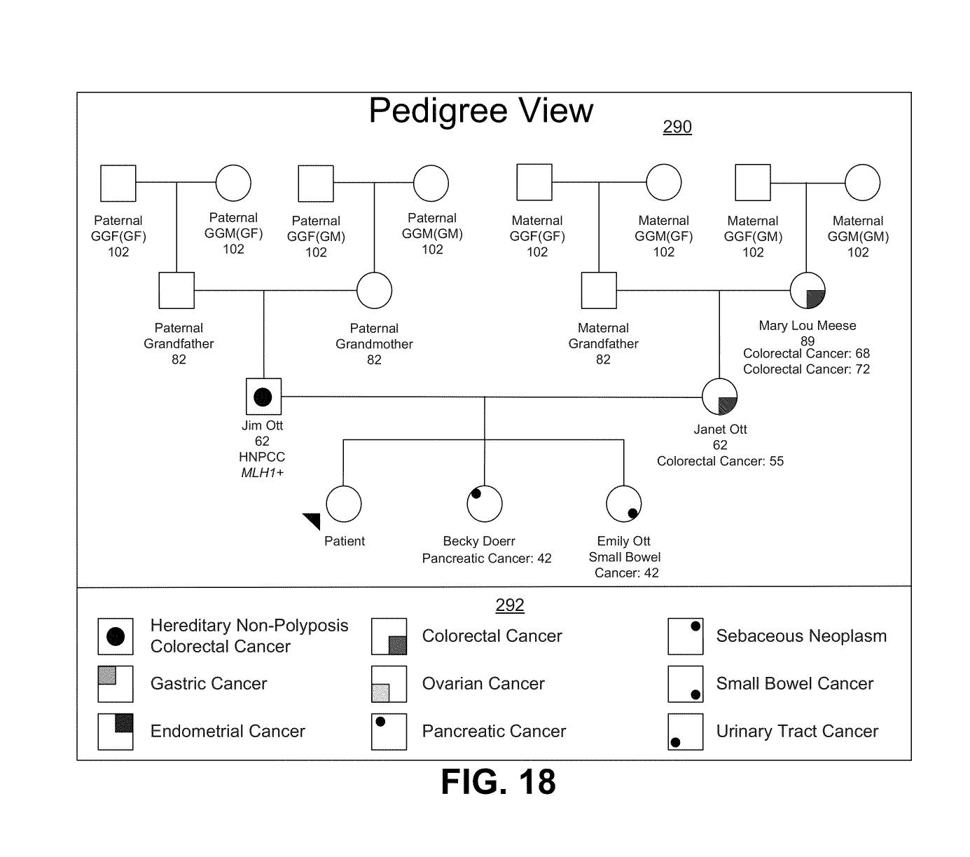

Furthermore, the clinician can view a disease overview page 270 as illustrated in FIG. 17 that displays annotated versions 272 of the clinical guidelines and published risk assessments that the interpreted statements and highest risk positively scored item modules that the disease specific risk reference is based on. A clinician can select a reason for review in choose reason for review section 276. After review of the disease specific risk reference page or pages by the clinician, the clinician can choose to accept the disease specific risk reference evaluation in a select an action section (266, 274) on either pages of FIG. 16 or 17, which results in the storing of the disease specific risk reference evaluation in an electronic health record database in an electronic health record system. Alternatively or additionally, the clinician can choose to have the disease specific risk reference reviewed by a genetic expert or counselor. Additionally, each page includes a pedigree section (268, 278) that a clinician can select from a variety of different pedigree disease specific risk references. In certain examples, the clinician may want to view the actual disease specific pedigree image. FIG. 18 illustrates a disease specific pedigree image 290 that corresponds to the responses and family structure information provided in answers to the example question of FIGS. 7-14. A legend 292 illustrates the various diseases across the family structure of the patient.

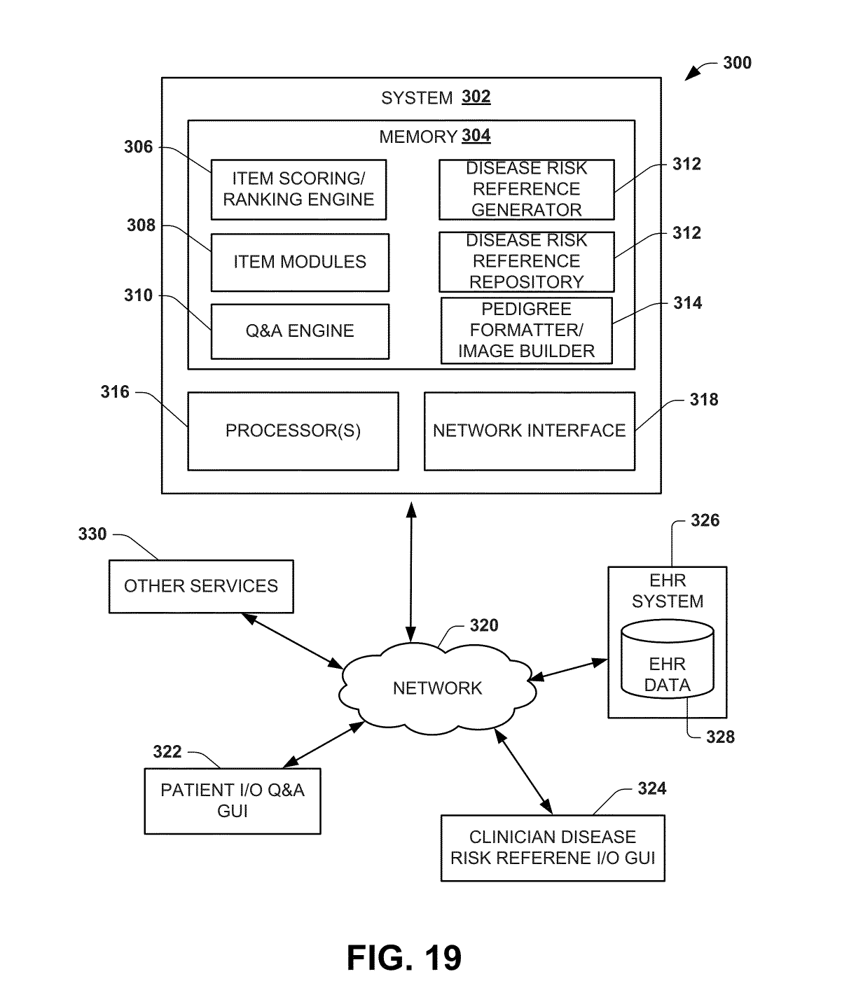

FIG. 19 depicts an example of a system architecture 300 in which a disease risk reference generation system 302 can be implemented. In the example of FIG. 19, the system 302 includes a memory 304 that includes machine readable instructions and data that can be utilized by the system 302 for implementing the functions and methods shown and described herein. For simplicity of explanation, the memory is 304 as depicted in FIG. 19 including an item scoring/ranking engine 306, a plurality of item modules 308, a Q&A engine 310, a disease risk reference generator 312, a disease risk decision repository 314 and a pedigree formatter/image builder 314. The system 302 also includes one or more processors 316 that can access the memory 304 and execute the associated instructions and utilize the data. The system 302 can also include a network interface 318 that can be utilized to access corresponding network 320. The network 320 can be implemented as including one or more local area network (LAN) or wide area network (WAN) or a combination of various networks. The network 320 may include wireless technology, fiber optic or electrically conductive medium for data communication.

The architecture 300 also employs one or more user interfaces at least including a patient I/O Q&A GUI 322 and a clinician disease risk reference I/O GUI 324 for reviewing one or more disease specific risk references. The user interfaces can be programmed for accessing the system 302 and implementing the functions and methods shown and described herein. For example, in response to a user input provided via the clinician disease risk reference I/O GUI 324, the one or more disease specific risk references can be provided to an EHR system 326 in which EHR data 328 is stored. Additionally, personal health questions, family structure questions and disease specific questions can be provided to patients and answers stored at the system 302 via the patient I/O Q&A GUI 322.

The system 302 can also communicate (e.g., retrieve and send) information relative to one or more other services 330. Such other services, for example, can include billing systems, insurance systems (internal to the organization or third party insurers), Personal Health Records, scheduling systems, prediction services, patient health portals or the like. In this way, the system can leverage information from a variety or resources and present users with current information that can be relevant to each patient or to groups of patients.

As will be appreciated by those skilled in the art, portions of the invention may be embodied as a method, data processing system, or computer program product. Accordingly, these portions of the present invention may take the form of an entirely hardware embodiment, an entirely software embodiment, or an embodiment combining software and hardware. Furthermore, portions of the invention may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention are described herein with reference to flowchart illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer, or other programmable data processing apparatus (or a combination of devices and circuits) to produce a machine, such that the instructions, which execute via the processor, implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks. The example systems and methods can be implemented as computer-readable instructions that can be stored in a non-transitory computer readable medium such as can be computer program product. The computer readable instructions corresponding can also be executed by one or more processors and/or across one or more computers.

What have been described above are examples. It is, of course, not possible to describe every conceivable combination of components or methodologies, but one of ordinary skill in the art will recognize that many further combinations and permutations are possible. Accordingly, the invention is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

1-26. (canceled)

27. A computer readable medium storing machine executable instructions, the machine executable instructions comprising:
an invitation question and answer engine configured to generate a questionnaire for a patient in response to a scheduled medical appointment and store responses to questions of the questionnaire in a memory as data associated with a plurality of item modules; and
an item scoring and ranking engine configured to score each of a subset of the item modules based on a contribution to a risk level for a disease associated with the corresponding item module and determine whether the score associated with one or more of the subset of the item molecules exceeds a threshold value indicating an increased risk of the disease.

28. The computer readable medium of claim 27, wherein the machine executable instructions further comprise a disease specific risk reference generator configured to extract from the memory item content associated with an item module with an associated score exceeding the threshold value.

29. The computer readable medium of claim 28, wherein the item content comprises a medication recommendation for the disease, a diagnosis code for the disease, an educational link for the disease, information related to an overview of the disease, or a pedigree chart related to the disease.

30. The computer readable medium of claim 27, wherein the data associated with an item module of the plurality of item modules comprises a logical Boolean operation based on a response to a question of the questionnaire.

31. The computer readable medium of claim 27, wherein the data associated with an item module of the plurality of item modules corresponds to at least two responses to at least two questions of the questionnaire.

32. The computer readable medium of claim 27 wherein the contribution to the risk level for the disease is based on a clinical guideline related to the disease or a published risk assessment related to the disease.

33. The computer readable medium of claim 27, wherein the invitation question and answer engine comprises a graphical user interface configured to display a portion of the questionnaire to the patient and receive a user input characterizing a response to the portion of the questionnaire from the patient.

34. The computer readable medium of claim 27, wherein the invitation question and answer engine is further configured to generate the questionnaire based on a question set stored in the memory and selected from a plurality of question sets based on the scheduled medical appointment.

35. The computer readable medium of claim 27, wherein the invitation question and answer engine is further configured to generate the questionnaire based on at least two question sets stored in the memory and selected from a plurality of question sets based on the scheduled medical appointment, and
wherein the invitation question and answer engine is further configured to suppress a redundant question in one of the at least two question sets.

36. The computer readable medium of claim 27, wherein the invitation question and answer engine is further configured to populate the questionnaire with a historical response to a question associated with a previous appointment request.

37. The computer readable medium of claim 27, wherein the invitation question and answer engine is further configured to generate supplemental questions for the questionnaire based on a user input characterizing a response from a patient to a portion of the questionnaire.

38. The computer readable medium of claim 37, wherein data based on the user input characterizing a response from the patient to the supplemental questions is associated with the same item module as the response to the portion of the questionnaire.

39. The computer readable medium of claim 27, wherein the questionnaire comprises at least one of a health history question set, a family structure question set, and a family disease history question set.

40. The computer readable medium of claim 27, wherein the plurality of item modules comprises at least one disease related item module and at least one family structure related item module.

41. A method, comprising:
generating, by one or more computing devices, a questionnaire for a patient in response to a scheduled medical appointment;
associating, by the one or more computing devices, data characterizing responses to questions of the questionnaire with plurality of item modules stored in memory;
scoring, by the one or more computing devices, each of a subset of the item modules based on a contribution to a risk level for a disease associated with the corresponding item module; and
determining, by the one or more computing devices, whether the score associated with one or more of the subset of the item molecules exceeds a threshold value indicating an increased risk of the disease.

42. The method of claim 41, further comprising extracting, by the one or more computing devices, item content associated with an item module with an associated score exceeding the threshold value from the memory.

43. The method of claim 42, wherein the item content comprises a medication recommendation for the disease, a diagnosis code for the disease, an educational link for the disease, information related to an overview of the disease, or a pedigree chart related to the disease.

44. The method of claim 41, wherein the data associated with each of the plurality of item modules comprises a logical Boolean operation based on one or more responses to the questionnaire.

45. The method of claim 41, wherein the data associated with at least one of the plurality of item modules is based on at least two responses to at least two questions of the questionnaire.

46. The method of claim 41, wherein the contribution to the risk level for the disease is based on a clinical guideline related to the disease or a published risk assessment related to the disease.

47. The method of claim 41, wherein the questionnaire is generated based on at least two question sets stored in the memory and selected from a plurality of question sets based on the scheduled medical appointment.

48. The method of claim 47, wherein the generating further comprises suppressing a redundant question in one of the at least two question sets.
